(19) United States
(12) Patent Application Publication
(10) Pub. No.: US 2008/0118450 A1
(43) Pub. Date: May 22, 2008

(54) Sunscreen lotions containing inorganic crystalline compounds that absorb ultraviolet and shortwave visible light

(76) Inventor: Robert D. Bibb, Myrtle Beach, SC (US)

Correspondence Address: Robert D. Bibb, 7554 Siena Blvd., Myrtle Beach, SC, 29572, US

(21) Appl. No.: 11/602,834

(22) Filed: Nov. 22, 2006

(51) Int. Cl. A61K 8/19; A61Q 17/04

(52) U.S. Cl. 424/59

(57) ABSTRACT

A sunscreen lotion employing a technology utilizing inorganic crystalline compounds that absorb ultraviolet light and short wave visible light and dissipate their absorbed energy as visible light.

### FIELD OF THE INVENTION

The present invention relates generally to the use of sunscreening agents such as lotions or creams applied to the surface of human skin to prevent ultraviolet light and short wave visible light induced damage. In particular, this invention relates to the novel use of inorganic crystalline compounds that absorb ultraviolet and short wave visible light and dissipate their absorbed energy by emitting wave lengths of visible light in a process called luminescence. These absorbance compounds comprise a diverse group of crystalline compounds including alkaline earth elements which contain but are not limited to magnesium, barium, aluminum and silicone.

### BACKGROUND OF THE INVENTION

The use of chemical sunscreens to block ultraviolet B rays (290 nm-320 nm) has existed for the past thirty-five years. More recent research and epidemiological studies have focused on the influence of ultraviolet light A (320 nm-400 nm) and visible light (400 nm-430 nm) on the development of melanoma and skin photo-aging. The use of ultraviolet B sunscreens have allowed humans to spend more time in the sun without developing painful ultraviolet light burns. This increased time in the sun has allowed much longer exposure to ultraviolet light A with the probable consequence of increased incidence of melanoma and photo-aging. Use of ultraviolet A blocking chemicals such as avobenzone and mexoryl are useful but imperfect. The critical wavelength at which their absorption of ultraviolet drops off significantly is approximately 385 nm. No existing sunscreen agents block short-wave visible light. The gap between 385 nm and 430 nm represents a significant defect in the energy absorption range of existing ultraviolet chemical sunscreen agents. When existing ultraviolet light chemical compounds absorb energy, they themselves are activated and may transfer energy to the skin-sunscreen interface. This energy transfer could have adverse consequences on the skin. Sunscreen lotions containing inorganic crystalline compounds containing that absorb ultraviolet and visible light offer distinct advantages over existing sunscreen lotions. They emit the energy absorbed back to their environment in the form of visible light in a process known as luminescence. This process does not harm the skin. These new sunscreen compounds are non-toxic, not absorbed by the skin and absorb in the wave-length spectrum of 230 nm to 450 nm.

### SUMMARY OF THE INVENTION

According to its major aspects and briefly stated, the present invention is a new sunscreening technique employing the use of inorganic crystalline compounds containing alkaline earth elements including but not limited to magnesium, barium, aluminum and silicone incorporated into appropriate vehicles for application to human skin. The present invention represents a significant departure from existing sunscreen agents in that it does not make the use of organic compounds. This new sunscreen converts excess energy to visible light that cannot harm human skin. It is non-toxic. It is not absorbed. It absorbs light in the UVB (290 nm-320 nm) and UVA (320-400) range as well as visible light in the range (400 nm-450 nm).

### DETAILED DESCRIPTION OF THE PREFERRED EMBODIMENTS

The present invention is a new sunscreen employing the use of all concentrations of inorganic crystalline compounds containing alkaline earth elements that absorb ultraviolet and short wave visible light in oil in water combinations, water in oil combinations and all possible aqueous mixtures. The new sunscreen will employ the use of all possible particle sizes of inorganic alkaline earth elements. The new sunscreen will employ all possible methods of suspending the active particles by the methods mentioned herein.

What is claimed:

1. A new sunscreen lotion employing the use of inorganic crystalline compounds that absorb ultraviolet and short wave visible light.

2. The sunscreen lotion recited in claim 1 comprises all possible concentrations of the inorganic crystalline compounds that absorb as stated herein.

3. The sunscreen lotion recited in claim 1 comprises all possible combinations water in oil, oil in water and other materials capable of suspending the active ingredient.

4. The sunscreen lotion recited in claim 1 comprises all particle sizes of the inorganic crystalline compounds stated herein.

5. The sunscreen lotion recited in claim 1 has ultraviolet light B, ultraviolet light A and short wave visible light absorbing capacity.

6. The sunscreen lotion recited in claim 1 dissipates energy in the form of visible light which is harmless to the skin.
